(19) United States
(12) Patent Application Publication
(10) Pub. No.: US 2026/0199244 A1
(43) Pub. Date: Jul. 16, 2026

(54) FORMULATION AND USE OF A FUSION PROTEIN

(71) Applicant: Daocheng ZHU, Shanghai (CN)

(72) Inventor: Daocheng ZHU, Shanghai (CN)

(21) Appl. No.: 19/136,723

(22) PCT Filed: Dec. 7, 2023
(86) PCT No.: PCT/CN2023/137153

§ 371 Date: Jun. 6, 2025

(30) Foreign Application Priority Data
Dec. 7, 2022 (WO) PCT/CN2022/137279

(51) Int. Cl. A61K 9/16; A61K 9/00; A61K 38/17; A61P 37/08

(52) U.S. Cl. CPC A61K 9/1623; A61K 9/0019; A61K 9/0043; A61K 9/0073; A61K 38/1709; A61P 37/08

(57) ABSTRACT

The present invention pertains to the biomedical field, and particularly relates to a solid formulation of a fusion protein, said solid formulation consists of particulates comprising a fusion protein and at least one carrier, and a method of preparing the solid formulation. The present invention further pertains to a method of treating allergic rhinitis in an individual, the method comprises administering to the individual a therapeutically effective amount of the solid formulation of the fusion protein.

### TECHNICAL FIELD

The present invention pertains to the biomedical field, and particularly relates to a solid formulation of a fusion protein, a method of preparing the solid formulation, and a method of treating allergic rhinitis in an individual with the solid formulation.

### BACKGROUND OF THE INVENTION

The immune system is always working to fight off parasites, fungi, viruses and bacteria. However, sometimes the immune system will treat a harmless substance (called an allergen) as an unwanted invader and try to fight it. This overreaction of the body's immune system to a typically harmless substance is called an allergic reaction.

There is a limited number of biologically active large molecules for treating an allergic reaction. Omalizumab is the only antibody approved by FDA for targeting and blocking immunoglobin E (IgE).

Research studies have shown that, by conjugating FcεRI receptor and FcγRII receptor in a fusion protein, the fusion protein can adjust the IgE mediated signaling pathway, suppress the release of active medium, and can thus inhibit allergic reaction (WO2005085291A1).

However, the preservation and storage of the large proteins such as the fusion proteins is of concern due to highly degradative nature of such fusion proteins, which may result from chemical instability (e.g., any process which involves modification of the protein by bond formation or cleavage resulting in a new chemical entity) or physical instability (e.g., changes in the higher order structure of the protein). Chemical instability might result from racemization, hydrolysis, oxidation, or beta elimination and physical instability might be due to denaturation, aggregation, precipitation or adsorption. A marketable protein formulation must be safe to administer, remain physically, chemically, and biologically stable during the recommended shelf life.

Allergic rhinitis is also known as “hay fever”, and is an inflammation in the nose which occurs when the immune system overreacts to allergens in the air.

The invention surprisingly reveals that the solid formulation of the present application can be applied into mammals through nasal administration, preferably administrated through inhaling, and can effectively suppress or alleviate the symptoms of allergic rhinitis.

### SUMMARY OF THE INVENTION

It is an object of the invention to provide a solid formulation of the fusion protein, which is stable during the recommended shelf life, preferably up to one year at room temperature. Such solid formulation comprises substantially spherical particles of a fusion protein dispersed in at least one carrier, and can be applied through nasal administration, preferably administrated through inhaling.

It is another object of the invention to provide a method of preparing the solid formulation.

It is yet another object of the invention to provide a method of treating allergic rhinitis with the solid formulation.

In one aspect, the invention provides a solid formulation consists of particulates comprising a fusion protein and at least one carrier. Carriers suitable for the invention include polylol, carbohydrate and sugar. The carrier is selected from the group consisting of sucrose, trehalose, sorbitol, glycerol, mannitol, lactose, xylitol, arabitol, erythritol, lactitol, maltitol, glucose, raffinose, maltose, dextran, inositol or combination thereof.

In a preferred aspect, the particulates have the fusion protein and two or more carriers, preferably two carriers being lactose and mannitol.

Said fusion protein is a polypeptide comprising an amino acid sequence of SEQ ID No 2, or an amino acid sequence comprising a deletion(s), substitution(s), addition(s), or insertion(s) of one or several amino acid residues in the amino acid sequences of SEQ ID No 2.

In a preferred aspect, said fusion protein comprises one of the following sequences:
1.) DNA sequence of SEQ ID No. 1;
2.) Amino acid sequence of SEQ ID No. 2;
3.) A DNA sequence has an amino acid sequence at least 95% identical to SEQ ID No 2, and also encodes the same function protein sequence.

In a preferred aspect, sequence 1's DNA sequence is composed of 1665 base pair; the reading frame is from the 1st base pair to the 1665th base pair starting with the 5′ end.

Expansion in any fragment of said fusion protein is also covered in the extent of the fusion protein.

Said fusion protein may be those as disclosed in WO2005085291A1.

In a preferred aspect, the fusion protein comprises the amino acid sequence of SEQ ID No. 2 or is at least 60%, 70%, 80%, 90%, 95%, 96%, 97%, 98%, or 99% identical to SEQ ID No. 2.

In a preferred aspect, the fusion protein is FP4 having an amino acid sequence of SEQ ID No. 2.

The invention surprisingly reveals that the biological activity of the solid formulation does not vary substantially when stored at room temperature for one year. Furthermore, the solid formulation has substantially spherical shape and substantially uniform diameter, and is suitable for administration through inhaling.

In another aspect, the invention provides a method of treating allergic rhinitis, the method comprises administering to an individual a therapeutically effective amount of a solid formulation comprising the fusion protein.

In another aspect, the invention provides use of a solid formulation comprising the fusion protein for preparing a medicament for treating allergic rhinitis in an individual.

In a preferred aspect, the fusion protein comprises the amino acid sequence of SEQ ID No. 2 or is at least 60%, 70%, 80%, 90%, 95%, 96%, 97%, 98%, or 99% identical to SEQ ID No. 2.

In a preferred aspect, the fusion protein is FP4 having an amino acid sequence of SEQ ID No. 2.

In one aspect, the invention refers to a solid formulation of a fusion protein, wherein said solid formulation consists of particulates comprising a fusion protein and at least one carrier, and the fusion protein is a protein comprising the amino acid sequence of SEQ ID No. 2 or is at least 60%, 70%, 80%, 90%, 95%, 96%, 97%, 98%, or 99% identical to SEQ ID No. 2.

In a preferred aspect, the invention refers to a solid formulation, wherein said particulate comprises the fusion protein and one or more carriers.

In a preferred aspect, the invention refers to a solid formulation, wherein said carrier is selected from the group consisting of polylol, carbohydrate and sugar.

In a preferred aspect, the invention refers to a solid formulation, wherein said carrier is selected from the group consisting of sucrose, trehalose, sorbitol, glycerol, mannitol, lactose, xylitol, arabitol, erythritol, lactitol, maltitol, glucose, raffinose, maltose, dextran, inositol or combination thereof.

In a preferred aspect, the invention refers to a solid formulation, wherein said particulates have a 50% volume cumulative diameter (D50) of 5 μm or lower, preferably 4.5 μm or lower, more preferably 3.5 to 4.5 μm, and a 90% volume cumulative diameter (D90) of 35 μm or lower, preferably 30 μm or lower, more preferably 28 to 30 μm.

In another preferred aspect, the invention refers to a solid formulation, wherein said particulates have a 50% volume cumulative diameter (D50) of higher than 5 μm to 12 μm, preferably 8 to 12 μm, yet preferably 8 to 10 μm, more preferably about 9 μm or about 10 μm, most preferably about 10 μm, and a 90% volume cumulative diameter (D90) of 40 μm or lower, preferably 38 μm or lower, more preferably 34 to 36 μm.

In a preferred aspect, the invention refers to a solid formulation, wherein said solid formulation is substantially free from surfactant, and is preferably a neat formulation.

In a preferred aspect, the invention refers to a solid formulation, wherein the solid formulation has a shelf life of 6 months or longer, preferably a year or longer.

In a preferred aspect, the invention refers to a solid formulation, wherein the solid formulation is inhalable.

In a preferred aspect, the invention refers to a solid formulation, wherein the solid formulation comprises 0.1-5 wt %, preferably about 0.5-4 wt %, more preferably 1-4 wt % of the fusion protein.

In another aspect, the invention refers to a method of preparing the above solid formulation, said method comprises the following steps: spraying the suspension or solution of the fusion protein into a flow of liquid droplets, entraining the flow of liquid droplets within a flow of coolant to freeze the liquid droplets into frozen particulates, and drying such particulates to form said solid formulation.

In another aspect, the invention refers to a method of treating allergic rhinitis in an individual, the method comprises administering to the individual a therapeutically effective amount of the above solid formulation.

In a preferred aspect, the invention refers to a method of treating allergic rhinitis in an individual, wherein the solid formulation is administered through inhaling, preferably through a nasal spray device.

### BRIEF DESCRIPTION OF THE DRAWINGS

FIG. 1 shows the particle size of one exemplary solid formulation of the present invention.

FIG. 2 shows the particle size of another exemplary solid formulation of the present invention.

FIG. 3 shows the biological activity and stability of the fusion protein FP4 present in the solid formulation of the present invention.

FIG. 4 shows that the FP4 fusion protein blocked allergic reaction in the transgenic mice.

### DETAILED DESCRIPTION OF THE INVENTION

### Definitions

Unless otherwise defined below, all technical and scientific terms used herein have the same meanings as commonly understood by one of ordinary skill in the art. References to techniques used herein are intended to refer to techniques that are generally understood in the art, including those obvious changes or equivalent replacements of the techniques for those skilled in the art. While it is believed that the following terms are well understood by those skilled in the art, the following definitions are set forth to better explain the invention.

As used herein, the terms “including”, “comprising”, “having”, “containing” or “comprising”, and other variants thereof, are inclusive or open, and do not exclude other unlisted elements or method steps.

The term “amino acid” refers to any compound containing both an amino group and a carboxylic acid group. Although the amino group most commonly occurs at the position adjacent to the carboxy function, the amino group may be positioned at any location within the molecule. The amino acid may also contain additional functional groups, such as amino, thio, carboxyl, carboxamide, imidazole, etc. An amino acid may be synthetic or naturally occurring, and may be used in either its racemic or optically active (D-, or L-) forms, including various ratios of stereoisomers.

A “neat” formulation in accordance with the invention refers to a solid formulation consisting of particulate or powder that contains a fusion protein and at least one carrier (typically the fusion protein takes from 0.1-5% by weight of the total mass of the fusion protein and the at least one carrier), which solid formulation is substantially absent of additional excipients, i.e., contains less than about 1% by weight additional excipients.

A “rapid acting” powder or particulate is a powder or particulate that does not possess controlled or sustained release characteristics when administered by inhalation.

A formulation consisting of particulate or powder refers to a powder formulation that typically contains less than about 10% moisture, preferably less than about 7% moisture, more preferably contains less than about 5-6% moisture, and even more preferably contains less than about 3% moisture, and even more preferably contains less than about 2% moisture, and most preferably contains less than about 1% moisture, depending upon the type of active agent in the formulation.

An “inhalable” formulation that is “suitable for nasal delivery” refers to a formulation comprising solid (i.e., non-solution) particles that are capable of (i) being readily dispersed in or by an inhalation device and (ii) inhaled by a subject so that at least most of the particles reach the nasal cavity. Such a powder is considered to be “respirable” or “inhalable”.

“Free from surfactant” in the context of the present invention refers to a formulation comprising less than about 0.1% by weight of a surfactant.

When “about” is followed by a value or a ratio, it means such value or ratio ±10%, preferably ±5%, more preferably ±1%.

“Substantially” has the meaning generally known in the art. For example, “substantially absent of (free from) X” means that X is present in 10 wt % or lower, preferably 5 wt % or lower, more preferably 1 wt % or lower; “does not change substantially” means that the decomposition rate is 10% or lower, preferably 5% or lower, more preferably 1% or lower.

“50% volume cumulative diameter (D50) of m or lower” means particles with a diameter of m or lower takes 50 volume % of the total particles. Similar meanings apply to 10% volume cumulative diameter (D10), 90% volume cumulative diameter (D90), etc. Particle size as reported herein are determined by laser diffraction method, although any of commonly employed techniques can be used for measuring the particle size (e.g., electron microscopy, light scattering, centrifugal sedimentation).

The term “solvate” as used herein is a substance formed by combination, physical binding and/or solvation of a compound of the invention with a solvent molecule, such as a disolvate, a monosolvate or a hemisolvate, wherein the ratio of the solvent molecule to the compound of the invention is about 2:1, about 1:1 or about 1:2, respectively. This kind of physical bonding involves ionization and covalent bonding (including hydrogen bonding) in different degrees. In some cases (e.g., when one or more solvent molecules are incorporated into crystal lattice of crystalline solid), the solvate can be isolated. Thus, the solvate comprises both solution phase and isolatable solvates. The compounds of the invention may be in solvated forms with pharmaceutically acceptable solvents (such as water, methanol and ethanol), and the present application is intended to encompass both solvated and unsolvated forms of the compounds of the invention.

One type of solvate is a hydrate. “Hydrate” relates to a specific subset of solvates wherein the solvent molecule is water. Solvates generally function in the form of pharmacological equivalents. The preparation of solvates is known in the art, see for example, M. Caira et al, J. Pharmaceut. Sci., 93(3): 601-611 (2004), which describes the preparation of a solvate of fluconazole with ethyl acetate and water. Similar methods for the preparation of solvates, hemisolvates, hydrates and the like are described by van Tonder et al, AAPS Pharm. Sci. Tech., 5(1): Article 12 (2004) and A. L. Bingham et al, Chem. Commun. 603-604 (2001). A representative and non-limiting method for the preparation of solvate involves dissolving a compound of the invention in a desired solvent (organic solvent, water or a mixture thereof) at a temperature above 20° C. to about 25° C., and then the solution is cooled at a rate sufficient to form a crystal, and the crystal is separated by a known method such as filtration. Analytical techniques such as infrared spectroscopy can be used to confirm the presence of the solvent in the crystal of the solvate.

“Pharmaceutically acceptable carrier” or “carrier” or “excipient” in the context of the present invention refers to a diluent, adjuvant, excipient or vehicle together with which the therapeutic agent is administered, and which is suitable for contacting a tissue of human and/or other animals within the scope of reasonable medical judgment, and without excessive toxicity, irritation, allergic reactions, or other problems or complications corresponding to a reasonable benefit/risk ratio. Furthermore, “Pharmaceutically acceptable carrier” or “carrier” or “excipient” in the context of the present invention refers to an excipient that may optionally be included in the formulation of the invention, and taken into the nasal cavity or lung with no significant adverse toxicological effects to the subject, and particularly to the nasal cavity or lung of the subject.

The pharmaceutically acceptable carriers or excipients that can be used in the pharmaceutical formulation of the invention include, but are not limited to, sterile liquids such as water and oils, including those oils derived from petroleum, animals, vegetables or synthetic origins, for example, peanut oil, soybean oil, mineral oil, sesame oil, etc. Other pharmaceutical carriers or excipients include starch, glucose, lactose, sucrose, gelatin, maltose, chalk, silica gel, sodium stearate, glyceryl monostearate, talc, sodium chloride, skimmed milk powder, glycerin, propylene glycol, mannitol, water, ethanol and the like. The pharmaceutical formulation may further contain a pH buffering agent as needed. Examples of suitable pharmaceutically acceptable carriers are as described in Remington's Pharmaceutical Sciences (1990).

The solid formulation of the invention may act systemically and/or locally. For this purpose, they may be administered via a suitable route, for example by nasal, transmucosal, topical administration, or by inhalation.

For these routes of administration, the solid formulation of the invention may be administered in a suitable dosage form.

The dosage forms include, but are not limited to, pulvis or sprays.

The term “effective amount” as used herein refers to an amount of active ingredient that, after administration, will relieve to some extent one or more symptoms of the condition being treated.

As used herein, “individual” includes a human or a non-human animal. Exemplary human individual includes a human individual (referred to as a patient) suffering from a disease (such as the disease described herein) or a normal individual. “Non-human animal” in the present invention includes all vertebrates, such as non-mammals (e.g., birds, amphibians, reptiles) and mammals, such as non-human primates, domestic animals, and/or domesticated animals (e.g., sheep, dogs, cats, cows, pigs, etc.).

### Solid Formulations

In order to better treat nasal diseases, administration through inhaling may be advantageous, because in this way, the pharmaceutical agent can directly reach the nasal cavity or lung, has rapid on-set, increases the bioavailability, and decreases the dosage needed. Furthermore, administration through inhaling decreases systematic exposure, lessens side effects by reducing the toxicity of the pharmaceutical agent, and is more convenient to the subject.

However, it remains challenging to formulate a protein drug into a stable powder or particulate.

Freeze-drying and spray-drying are used in general to formulate protein drugs into powders. Freeze-drying is most widely employed nowadays. Freeze-drying is suitable for heat-sensitive proteins, but it is not suitable for producing a uniform powder with a diameter of several to several tons of micrometers, a form that can be readily inhaled. Freeze-drying also tends to concentrate proteins between the ice crystals while cooling. This concentration brings about a rapid change in the pH and ionic strength surrounding the protein to cause denaturation and precipitation (Schwartz, P. L. et al, Endocrinology, 92(6): 1795, 1973; Koseki, T. et al, J. Biochem., 107:389, 1990). In contrast, spray-drying involves spraying a continuous stream of a liquid sample to form microscopically dispersed droplets, while instantaneously drying them with hot air at the same time. Spray-drying has been in use for formulating various drugs. Spray-drying has the advantage of producing powders whose particle sizes are suitable for delivering drugs to respiratory tracts and lungs. Since proteins in general are not stable against heat, there are not many cases of protein drug formulations spray-dried with hot air.

Although some power or particulate solid formulations of proteins in the prior art do not vary in physical state (do not form lumps) at room temperature for a certain amount of time, it is most difficult for such solid formulation to retain the biological activity for a prolonged time period. Or, such solid formulation may not be in the particulate form, and may not be suitable for inhaling.

It is further found in present invention that the solid formulation having a D50 particle size of about 10 μm is most suitable for nasal administration for the treatment of allergic rhinitis. The solid formulation has a particle size of about 10 μm, e.g. 8 to 12 μm, 5 to 15 μm, or 2 to 20 μm, which is the optimum particle size to be adsorbed on nasal mucosa for increasing bioavailability of the fusion protein.

The present invention surprisingly reveals that the fusion protein can be dispersed well in lactose and mannitol, forming substantially uniform particulates, which are stable at room temperature, and the biological activity of the fusion protein contained therein does not change substantially for up to one year, and are especially suitable for administration though inhaling. The mass percentage of the fusion protein and the carriers such as lactose and mannitol can vary, as long as the fusion protein stays stable in the solid formulation.

In a preferred aspect, the solid formulation of the present invention is substantially free from surfactant, and is more preferably a neat formulation.

In a preferred aspect, the solid formulation of the present invention is prepared through an atmospheric pressure freeze-drying process.

In another aspect, the present invention provides a method of preparing the solid formulation of the fusion protein, said method comprises spraying the suspension or solution of the fusion protein into a flow of liquid droplets, entraining the flow of liquid droplets within a flow of coolant to freeze the liquid droplets into frozen particulates, and drying such particulates to form said solid formulation.

### Therapeutic Methods and Uses

The present invention reveals that the solid formulation comprising the fusion protein formed by conjugating FcεRI receptor and FcγRII receptor (preferably comprising the amino acid sequence of SEQ ID No. 2 or is at least 60%, 70%, 80%, 90%, 95%, 96%, 97%, 98%, or 99% identical to SEQ ID No. 2, more preferably the fusion protein is FP4 having an amino acid sequence of SEQ ID No. 2) is especially effective for treating allergic rhinitis. Preferably, said solid formulation of the present invention can be applied into mammals through nasal administration, preferably administrated through inhaling, and can effectively suppress or alleviate the symptoms of allergic rhinitis.

### EXAMPLES

In order to make the objects and technical solutions of the present invention clearer, the present invention will be further described below in conjunction with specific example. It should be understood that the examples are not intended to limit the scope of the invention. Further, specific experimental methods not mentioned in the following examples were carried out in accordance with a conventional experimental method.

### Material and Methods

The fusion protein having the amino acid sequence of SEQ ID No. 2 (called FP4) is prepared and purified according to WO2005085291A1.

Other reagents including mannitol, lactose, PBS buffer and Evans blue are purchased from Fisher Scientific.

The FcεRIα-transfected CHO3D10 cells are purchased from ATCC.

The Transgenic mice are purchased from Jackson Laboratory.

### Example 1: Preparation and Characterization of the Solid Formulation

FP4 is mixed well with mannitol and lactose in water, and the mixture is subjected to an atmospheric pressure freeze-drying process to yield the solid formulation. Specifically, the mixture is sprayed into a flow of liquid particles, the flow of liquid particles are entrained within a flow of coolant to form frozen particulates at a reduced temperature sufficient to form the frozen particulates, and such frozen particulates are dried at a relatively low temperature (can be higher than said reduced temperature) to form said solid formulation.

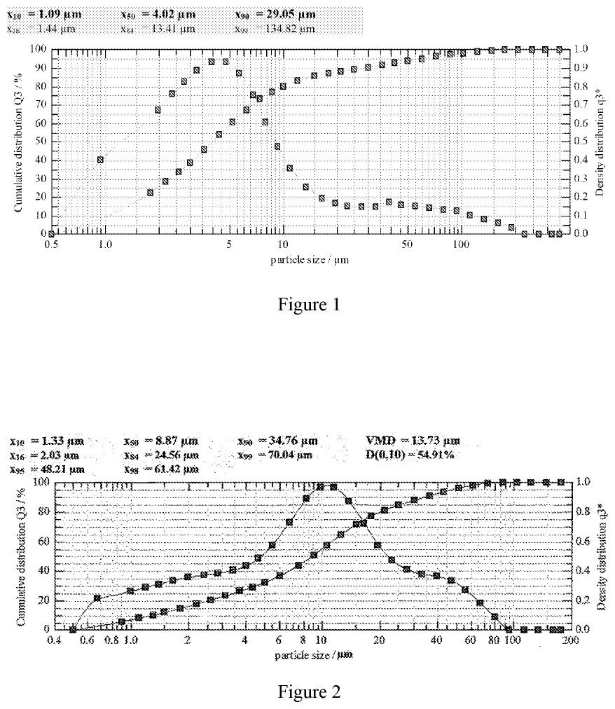

Use SympaTEC HELOS/BR device to measure the particle size of such particles, the exemplary results are as shown in FIG. 1. It can be seen that particles with a diameter of 1.09 μm or lower takes 10 volume %, particles with a diameter of 1.44 μm or lower takes 16 volume %, particles with a diameter of 4.02 μm or lower takes 50 volume %, particles with a diameter of 13.41 μm or lower takes 84 volume %, particles with a diameter of 29.05 μm or lower takes 90 volume %, and particles with a diameter of 134.82 μm or lower takes 99 volume %.

Multiple experiments were performed as duplicates. The particle size distribution of another exemplary result is as shown in FIG. 2. It can be seen that particles with a diameter of 1.33 μm or lower takes 10 volume %, particles with a diameter of 2.03 μm or lower takes 16 volume %, particles with a diameter of 8.87 μm or lower takes 50 volume %, particles with a diameter of 24.56 μm or lower takes 84 volume %, particles with a diameter of 34.76 μm or lower takes 90 volume %, particles with a diameter of 48.21 μm or lower takes 95 volume %, particles with a diameter of 61.42 μm or lower takes 98 volume %, and particles with a diameter of 70.04 μm or lower takes 99 volume %. As compared to the results shown in FIG. 1, the median particle size is shifted to a relatively higher value and the distribution has a relatively narrower span.

### Example 2: Activity and Stability Study of the Solid Formulation

The solid formulation is stored at room temperature for one month. The biological activity of the solid formulation is measured according to the following procedure.

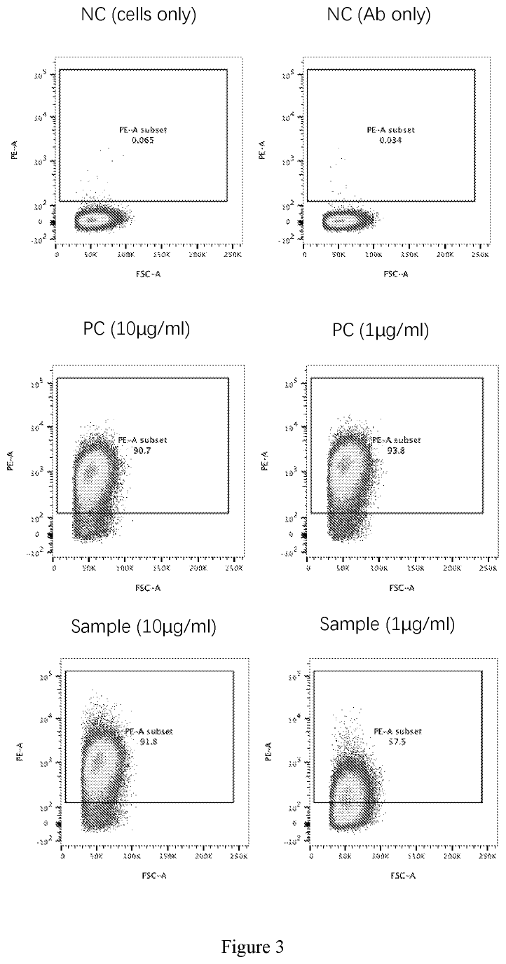

The solid formulation as prepared in Example 1 with a D50 of 8.87 μm and as stored above was dissolved in PBS buffer and then incubated with human FcεRIα-transfected CHO3D10 cells. After 1 hour, the cells were washed and stained with PE-conjugated anti-human IgE. The binding ability was analyzed by flow cytometry (see the “sample” row in FIG. 3). The purified FP4 protein was used as positive control (see the “PC” row in FIG. 3). The cells only and the antibody (“Ab”) only were used as the negative control (see the “NC” row in FIG. 3).

FIG. 3 indicates that, the biological activity of FP4 does not change substantially when the solid formulation comprising FP4 is stored at room temperature for one month.

After the solid formulation is stored at room temperature for longer time periods such as two months, three months . . . up to one year, the biological activity of the solid formulation is measured according to the above procedure, and the test results indicate that the biological activity of FP4 does not change substantially when the solid formulation comprising FP4 is stored at room temperature for up to one year.

### Example 3: Biological Study of the Solid Formulation

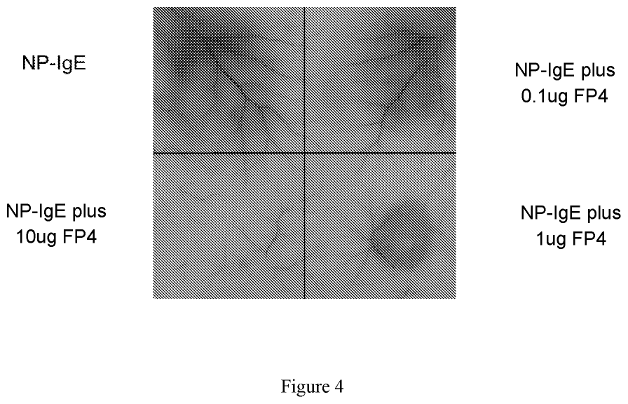

Transgenic mice expressing the human FcεRIα chain and with the murine FcεRIα chain knocked out were primed intradermally with 250 ng of NP-specific recombinant human IgE in 50 μl saline. Individual sites were injected with different doses of FP4 fusion protein simultaneously. 4 h later, the mice were then given an intravenous challenge with 1.5 mg/ml of NP-BSA plus 1% Evans blue in 300 μl saline solution. Cutaneous anaphylaxis was assessed visually by the blue dye leakage from blood vessels into the skin. If allergic reaction occurs, the local skin color turns to blue because of the leakage of dye from blood vessel. As shown in FIG. 4, when the FP4 fusion protein was added, the allergic reaction was blocked to some extent for the 0.1 μg and 1 μg FP4 fusion protein, and was completely blocked for the 10 μg FP4 fusion protein (the local skin color does not present blue). This experimental result further proved that the FP4 fusion protein in vivo inhibited allergic reaction.

### Example 4: Treatment of Allergic Rhinitis with the Solid Formulation

Volunteers diagnosed with allergic rhinitis participate in the test of treatment effect of the solid formulation of the present invention. 50 volunteered subjects with different demographic characteristics such as gender, age, weight were involved in the test. The participants self-evaluate their nasal symptoms according to the following criteria in Table 1 below considering the symptoms of sneezing, runny nose, stuffy nose and itchy nose. A score of 0, 1, 2 or 3 was given by each of the participants themselves before and after the treatment.

TABLE 1
Criteria for symptoms of allergic rhinitis

| | Severity (score) | | | |
|---|---|---|---|---|
| Symptom | None (0) | Mild (1) | Moderate (2) | Severe (3) |
| Sneezing | No sign or | Signs/symptoms | Clearly feels | Unable to tolerate |
| Runny nose | symptom | exists, but feels | signs/symptoms, | signs/symptoms |
| Stuffy nose | | mild and easily | suffering but | and daily life or |
| Itchy nose | | tolerable | tolerable | sleep was disrupted |

During treatment, each participant was treated with 2 mg of the solid formulation as prepared in Example 1 with a D50 of 8.87 μm (the FP4 fusion protein takes around 1 wt % of the total mass, i.e. a dosage of about 20 μg) through inhaling for each nostril each time, 2 times a day, lasting for 3 days. The scores reported by the participant before and after the treatment were listed in following Table 2.

TABLE 2
Comparison of nasal symptom scores before and after
treatment of solid formulation of fusion protein

| | Before | After |
|---|---|---|
| # | treatment | treatment |
| JZ -2001 | 3 | 1 |
| JZ -2002 | 3 | 0 |
| JZ -2003 | 3 | 1 |
| JZ -2004 | 3 | 1 |
| JZ -2005 | 3 | 1 |
| JZ -2006 | 3 | 0 |
| JZ -2007 | 3 | 0 |
| JZ -2008 | 3 | 0 |
| JZ -2009 | 2 | 0 |
| JZ -2010 | 3 | 1 |
| JZ -2011 | 2 | 0 |
| JZ -2012 | 3 | 0 |
| JZ -2013 | 3 | 1 |
| JZ -2014 | 3 | 0 |
| JZ -2015 | 3 | 0 |
| JZ -2016 | 3 | 1 |
| JZ -2017 | 3 | 0 |
| JZ -2018 | 3 | 1 |
| JZ -2019 | 2 | 0 |
| JZ -2020 | 3 | 1 |
| JZ -2021 | 3 | 0 |
| JZ -2022 | 3 | 0 |
| JZ -2023 | 3 | 0 |
| JZ -2024 | 2 | 0 |
| JZ -2025 | 3 | 1 |
| JZ -2026 | 3 | 1 |
| JZ -2027 | 3 | 0 |
| JZ -2028 | 3 | 1 |
| JZ -2029 | 3 | 0 |
| JZ -2030 | 3 | 0 |
| JZ -2031 | 3 | 1 |
| JZ -2032 | 3 | 0 |
| JZ -2033 | 2 | 0 |
| JZ -2034 | 3 | 1 |
| JZ -2035 | 3 | 1 |
| JZ -2036 | 3 | 0 |
| JZ -2037 | 3 | 0 |
| JZ -2038 | 3 | 1 |
| JZ -2039 | 2 | 0 |
| JZ -2040 | 3 | 1 |
| JZ -2041 | 3 | 1 |
| JZ -2042 | 3 | 0 |
| JZ -2043 | 3 | 1 |
| JZ -2044 | 3 | 0 |
| JZ -2045 | 3 | 1 |
| JZ -2046 | 2 | 0 |
| JZ -2047 | 3 | 0 |
| JZ -2048 | 3 | 1 |
| JZ -2049 | 2 | 0 |
| JZ -2050 | 3 | 1 |
| Average | 2.84 | 0.44 |

As can be seen from the above results, after the treatment, volunteers are observed to have significantly alleviated symptoms of stuffy nose, sneezing and/or runny nose, or these symptoms are fully recovered.

### Abbreviations and Specialist Terms

| | |
|---|---|
| mg/kg | Milligram per kilogram |
| n | Number |
| PBS | Phosphate buffer saline |
| NP | 4-hydroxy-3-nitrophenylacetyl |

Sequence Listing

SEQ ID NO: 1
ttcaccccgcccaccgtgaagatcttacagtcgtcctgcgacggcggcgggcacttccccccgaccatccagctc
ctgtgcctcgtctctgggtacaccccagggactatcaacatcacctggctggaggacgggcaggtcatggacgtg
gacttgtccaccgcctctaccacgcaggagggtgagctggcctccacacaaagcgagctcaccctcagccagaa
gcactggctgtcagaccgcacctacacctgccaggtcacctatcaaggtcacacctttgaggacagcaccaagaa
gtgtgcagattccaacccgagaggggtgagcgcctacctaagccggcccagcccgttcgacctgttcatccgcaa
gtcgcccacgatcacctgtctggtggtggacctggcacccagcaaggggaccgtgaacctgacctggtcccggg
ccagtgggaagcctgtgaaccactccaccagaaaggaggagaagcagcgcaatggcacgttaaccgtcacgtc
caccctgccggtgggcacccgagactggatcgagggggagacctaccagtgcagggtgacccacccccacct
gcccagggccctcatgcggtccacgaccaagaccagcggcccgcgtgctgccccggaagtctatgcgtttgcga
cgccggagtggccggggagccgggacaagcgcaccctcgcctgcctgatccagaacttcatgcctgaggacat
ctcggtgcagtggctgcacaacgaggtgcagctcccggacgcccggcacagcacgacgcagccccgcaagac
caagggctccggcttcttcgtcttcagccgtctagaggtgaccagggccgaatgggagcagaaagatgagttcat
ctgccgtgcagtccatgaggcagctagcccctcacagaccgtccagcgagcggtgtctgtaaatcccggtaaag
gatccgagcccaaatcttgtgacaaaactcacacatgcccaccgtgcccagcacctgaactcctggggggaccgt
cagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaaga
caaagccgcgggaggagcagtacaacagcacgtaccgggtggtcagcgtcctcaccgtcctgcaccaggactg
gctgaatggcaaggagtacaagtgcaaggtctccaacaaagccctcccagcccccatcgagaaaaccatctcca
aagccaaagggcagccccgagaaccacaggtgtacaccctgcccccatcccgggatgagctgaccaagaacc
aggtcagcctgacctgcctggtcaaaggcttctatcccagcgacatcgccgtggagtgggagagcaatgggcag
ccggagaacaactacaagaccacgcctcccgtgctggactccgacggctccttcttcctctacagcaagctcacc
gtggacaagagcaggtggcagcaggggaacgtcttctcatgctccgtgatgcatgaggctctgcacaaccactac
acgcagaagagcctctccctgtctccgggtaaatga
SEQ ID NO: 2
FTPPTVKILQSSCDGGGHFPPTIQLLCLVSGYTPGTINITWLEDGQVMDV
DLSTASTTQEGELASTQSELTLSQKHWLSDRTYTCQVTYQGHTFEDSTK
KCADSNPRGVSAYLSRPSPFDLFIRKSPTITCLVVDLAPSKGTVNLTWSR
ASGKPVNHSTRKEEKQRNGTLTVTSTLPVGTRDWIEGETYQCRVTHPHL
PRALMRSTTKTSGPRAAPEVYAFATPEWPGSRDKRTLACLIQNFMPEDIS
VQWLHNEVQLPDARHSTTQPRKTKGSGFFVFSRLEVTRAEWEQKDEFIC
RAVHEAASPSQTVQRAVSVNPGKGSEPKSCDKTHTCPPCPAPELLGGPSV
PLPPPKPKDTLMISRTPEVTCVVVDVSHEDPEVKPNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGPYPSDIAVEWESNGQP
ENNYKTTPPVLDSDGSPPLYSKLTVDKSRWQQGNVPSCSVMHEALHNH
YTQKSLSLSPGK

1. A solid formulation of a fusion protein, wherein said solid formulation consists of particulates comprising a fusion protein and at least one carrier, and the fusion protein is a protein comprising the amino acid sequence of SEQ ID No. 2 or is at least 60%, 70%, 80%, 90%, 95%, 96%, 97%, 98%, or 99% identical to SEQ ID No. 2.

2. The solid formulation according to claim 1, wherein the fusion protein is FP4 having an amino acid sequence of SEQ ID No. 2.

3. The solid formulation according to claim 1, wherein said particulate comprises the fusion protein and one or more carriers.

4. The solid formulation according to claim 1, wherein said carrier is selected from the group consisting of polylol, carbohydrate and sugar.

5. The solid formulation according to claim 1, wherein said carrier is selected from the group consisting of sucrose, trehalose, sorbitol, glycerol, mannitol, lactose, xylitol, arabitol, erythritol, lactitol, maltitol, glucose, raffinose, maltose, dextran, inositol or combination thereof.

6. The solid formulation according to claim 1, wherein said particulates have a 50% volume cumulative diameter (D50) of 5 μm or lower, preferably 4.5 μm or lower, more preferably 3.5 to 4.5 μm, and a 90% volume cumulative diameter (D90) of 35 μm or lower, preferably 30 μm or lower, more preferably 28 to 30 μm.

7. The solid formulation according to claim 1, wherein said particulates have a 50% volume cumulative diameter (D50) of higher than 5 μm to 12 μm, preferably 8 to 12 μm, yet preferably 8 to 10 μm, more preferably about 9 μm to about 10 μm, most preferably about 10 μm, and a 90% volume cumulative diameter (D90) of 40 μm or lower, preferably 38 μm or lower, more preferably 34 to 36 μm.

8. The solid formulation according to claim 1, wherein said solid formulation is substantially free from surfactant, and is preferably a neat formulation.

9. The solid formulation according to claim 1, wherein the solid formulation has a shelf life of 6 months or longer, preferably a year or longer.

10. The solid formulation according to claim 1, wherein the solid formulation is inhalable.

11. The solid formulation according to claim 1, wherein the solid formulation comprises 0.1-5 wt %, preferably about 0.5-4 wt %, more preferably 1-4 wt % of the fusion protein.

12. A method of preparing a solid formulation according to claim 1, said method comprises the following steps: spraying the suspension or solution of the fusion protein into a flow of liquid droplets, entraining the flow of liquid droplets within a flow of coolant to freeze the liquid droplets into frozen particulates, and drying such particulates to form said solid formulation.

13. A method of treating allergic rhinitis in an individual, the method comprises administering to the individual a therapeutically effective amount of the solid formulation according to claim 1.

14. The method according to claim 13, wherein the solid formulation is administered through inhaling, preferably through a nasal spray device.


SEQUENCE LISTING (2 sequences)

SEQ ID NO: 1 - DNA, 1665 bp, synthetic construct
  source 1..1665: mol_type = other DNA
ttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgtctagaggtgaccagggccgaatgggagcagaaagatgagttcatctgccgtgcagtccatgaggcagctagcccctcacagaccgtccagcgagcggtgtctgtaaatcccggtaaaggatccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 554 aa, synthetic construct
FTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKGSEPKSCDKTHTCPPCPAPELLGGPSVPLPPPKPKDTLMISRTPEVTCVVVDVSHEDPEVKPNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGPYPSDIAVEWESNGQPENNYKTTPPVLDSDGSPPLYSKLTVDKSRWQQGNVPSCSVMHEALHNHYTQKSLSLSPGK